(19) United States
(12) Patent Application Publication
(10) Pub. No.: US 2009/0060791 A1
(43) Pub. Date: Mar. 5, 2009

(54) MICROCHANNEL CHIP AND METHOD FOR MANUFACTURING SUCH CHIP

(75) Inventors: Hisashi Hagiwara, Kanagawa (JP); Yoshinori Mishina, Kanagawa (JP); Toshiharu Shiraishi, Kanagawa (JP)

Correspondence Address: DARBY & DARBY P.C., P.O. BOX 770, Church Street Station, New York, NY, 10008-0770, US

(73) Assignee: Aida Engineering, Ltd., Sagamihara-shi (JP)

(21) Appl. No.: 12/279,241

(22) PCT Filed: Feb. 9, 2007
(86) PCT No.: PCT/JP2007/052341

§ 371 Date: Aug. 13, 2008

(30) Foreign Application Priority Data
Feb. 15, 2006 (JP) 2006-037946

(51) Int. Cl. B01J 19/00; C12M 1/34; B05D 1/32

(52) U.S. Cl. 422/68.1; 435/287.1; 427/282

(57) ABSTRACT

The present invention provides a microchannel chip having microchannels fabricated without using an original such as a mold. The microchannel chip of the present invention comprises at least an upper substrate and a lower substrate, the upper substrate and the lower substrate being bonded together, characterized in that at least one non-bonding thin-film layer is formed on the bonding side of at least one substrate and that opposite ends of the non-bonding thin-film layer are each connected to a port open to the atmosphere. When a positive pressure is applied through one port, the area that corresponds to the non-bonding thin-film layer inflates to create a gap that can function as a microchannel, with the result that a liquid and/or a gas can be transferred from one port to the other.

### CROSS-REFERENCE TO PRIOR APPLICATION

This is a U.S. national phase application under 35 U.S.C. § 371 of International Patent Application No. PCT/JP2007/052341 filed Feb. 9, 2007 which claims the benefit of Japanese Patent Application No. 2007-037946 filed Feb. 15, 2006, both of which are incorporated by reference herein. The International Application was published in Japanese on Aug. 23, 2007 as WO 2007/094254 A1 under PCT Article 21(2).

### TECHNICAL FIELD

The present invention relates to a microchannel chip and a process for producing the same. More particularly, it relates to a microchannel chip in which a microchannel or microchannels serving as passages for a medium such as a liquid or gas can be formed without using an original such as a mold; it also relates to a process for producing the microchannel chip.

### BACKGROUND ART

Devices commonly known as “micro-total analysis systems (μ TAS)” or “lab-on-chip” include a substrate and microstructures such as microchannels and ports that are provided in the substrate to form channels of specified shapes. It has recently been proposed that a variety of operations such as chemical reaction, synthesis, purification, extraction, generation and/or analysis be performed on substances in the microstructures. Structures that are fabricated for this purpose and which have microstructures such as microchannels and ports provided in the substrate are collectively referred to as “microchannel chips” or “micro-fluid devices.”

Microchannel chips find use in a wide range of applications including gene analysis, clinical diagnosis, drug screening and environmental monitoring. Compared to devices of the same type in usual size, microchannel chips have various advantages including (1) extremely smaller amounts of samples and reagents that need to be used, (2) shorter analysis time, (3) higher sensitivity, (4) portability to the site for on-site analysis, and (5) one-way use.

A conventional microchannel chip is shown in FIGS. 8A and 8B, where it is indicated by numeral 100. As shown, the microchannel chip 100 comprises an upper substrate 102 formed of a material such as a synthetic resin, at least one microchannel 104 formed in the upper substrate 102, a port 105 or 106 formed at least one end of the micro-channel 104 to serve as an input or output port, and a lower substrate 108 that is bonded to the lower side of the substrate 102 and which is formed of a transparent or opaque material (for example, glass or a synthetic resin film). The lower substrate 108 helps seal the bottoms of the ports 105 and 106, as well as the micro-channel 104.

The materials and structures of microchannel chips of the type shown in FIGS. 8A and 8B, as well as processes for producing them may be found in JP 2001-157855 A, U.S. Pat. No. 5,965,237, and David C. Duffy et al., Rapid Prototyping of Microfluidic Systems in Poly(dimethylsiloxane), Analytical Chemistry, Vol. 70, No. 23, Dec. 1, 1988, pp. 4974-4984. Developed among them are a series of microchannel chips featuring the use of polydimethyl siloxane (PDMS). PDMS has good mold transferability to masters (molds) having channels and other microstructures, as well as high transparency, chemical resistance, and biocompatibility, thus having particularly outstanding features as constituent materials for microchannel chips.

FIG. 9 is a flowchart illustrating an exemplary process for producing the microchannel chip 100 shown in FIGS. 8A and 8B. This process is based on the so-called lithographic technology which is extensively used in the manufacture of semiconductors. First, in step (a), a silicon wafer 200 is provided which is of generally the same size as the final product microchannel chip (measuring, for example, 20 mm×20 mm or 20 mm×30 mm). The silicon wafer 200 may be subjected to a desired preliminary treatment such as drying or surface treatment. Thereafter, in step (b), a suitable resist material (e.g., negative photoresist SU-8) is applied by spin coating at a rate of 2000 rpm to 5000 rpm for several seconds to several tens of seconds, then dried in an oven to form a desired thickness of resist film 220. Subsequently, in step (c), the resist film 220 is exposed to a suitable exposing apparatus (not shown) through a mask 240. The mask 240 has a layout pattern corresponding to the channel 104 in the microchannel chip 100. Thereafter, in step (d), development is performed in a suitable liquid developer (e.g., 1-methoxy-2-propylacetic acid) to form a master (mold) 280 having a microstructure 260 corresponding to the channel 104 on the upper surface. If desired, the master 280 may be washed with an organic solvent (e.g., isopropyl alcohol) and distilled water. Further, the surface of the master 280 may be treated with a reactive ion etching system in the presence of trifluoromethane. The reactive ion etching treatment in the presence of trifluoromethane improves the mold release of PDMS from the master 280 in a later step. Subsequently, in step (e), a PDMS prepolymer mixed solution prepared by mixing a PDMS prepolymer and a curing agent in suitable proportions and deaerating the mixture is poured onto the upper surface of the master 280. In this case, a frame is preferably used as a casting mold, into which the PDMS prepolymer mixed solution is poured for templating. A suitable example of the PDMS prepolymer mixed solution that can be used is SYLGARD 184 SILICONE ELSASTOMER of Dow Corning, USA. This is a mixture of a liquid PDMS prepolymer and a curing agent in a ratio of 10:1. After the application, the coating may be left at ordinary temperatures for a sufficient time to cure or, alternatively, it may be heated, typically in an oven, at 65° C. for 1 hour or at 135° C. for 15 minutes, to thereby generate an intermediate PDMS substrate 300. The intermediate PDMS substrate 300 is a highly transparent rubbery resin, to which the microstructure 260 of the master 280 has been transferred. Thereafter, in step (f), the PDMS intermediate substrate 300 is stripped from the master 280, and a port 105 (106) is bored through the PDMS intermediate substrate 300 by means of a punch 320 so as to establish communication between its upper surface and the underlying hollow microchannel 104 to thereby obtain a PDMS substrate 102. Subsequently, in step (g), the PDMS substrate 102 is attached to an opposing substrate 108, with the side where the channel 104 is formed facing down. Finally, in step (h), the completed microchannel chip 100 is recovered.

However, in order to implement the lithographic technology as depicted in FIG. 9, an exposing mask must first be prepared in order to fabricate the master (mold) that serves as the original. To prepare the mask, an expensive production apparatus must be employed. Further, in order to expose the resist through the mask, an expensive exposing apparatus must be employed. In addition, not only a developing apparatus is necessary after exposure but also the spent liquid developer needs to be disposed of by some treatment. Therefore, the fabrication of the master (template) 280 involves extremely huge amounts of labor, time and cost, contributing to an increased cost of the microchannel chip 100 as the final product. What is more, in the case of a resist mold, its rigidity has led to poor durability and adhesion, with the result that it breaks fairly easily. Hence, it has been required that each time it breaks, the resist-made master (mold) 280 be remade by the above-described procedure. This has resulted in ever increasing costs for the production of the microchannel chip 100, thus making it difficult to supply disposable chips at lower cost.

In the case of feeding a medium such as a liquid from the port 105 to the port 106, a fluid control mechanism such as a micro-valve is sometimes provided halfway down the hollow microchannel 104 in order to control the flow of the medium (see, for example, JP 2001-304440 A, FIG. 3). However, the micro-valve is so complicated in structure that it is not easy to form and if it is to be actually installed, the manufacturing cost of the microchannel chip 100 is all the more increased.

### SUMMARY OF THE INVENTION

It is, therefore, an object of the present invention to provide a microchannel chip having a microchannel or microchannels fabricated without using an original such as a mold.

Another object of the present invention is to provide a process by which a microchannel chip having a microchannel or microchannels that should serve as passages for a medium such as a liquid or gas can be formed without using an original such as a mold.

As a means for solving the first-mentioned object, the invention provides a microchannel chip having at least an upper substrate and a lower substrate, the upper substrate and the lower substrate being bonded together, wherein at least one non-bonding thin-film layer is formed on the bonding side of at least one substrate and at least one end portion of the non-bonding thin-film layer is connected to a port open to the atmosphere.

According to this invention, by applying a positive pressure via the port, the area that corresponds to the non-bonding thin-film layer inflates to create a gap that can function as a microchannel. Consequently, a liquid and/or a gas can be forced from one port into the gap that has been created by the inflating. If both ends of the non-bonding thin-film layer are connected to ports open to the atmosphere, a liquid and/or a gas can be transferred from one port to the other. And depending on the mode of use, the area that corresponds to the non-bonding thin-film layer can fulfill the function of an on-off valve or a micro-valve.

As a means for solving the first-mentioned object, the invention can be further characterized in that the non-bonding thin-film layer further includes, halfway down it, at least one layer of enlarged region having at least one planar shape selected from the group consisting of a circular, an elliptical, a rectangular, and a polygonal shape.

According to this invention, the layer of enlarged region, when inflated, can function as a liquid reservoir, which liquid reservoir portion can be utilized to ensure efficient performance of PCR amplification and other operations.

As a means for solving the first-mentioned object, the invention recited above provides a microchannel chip according to claim 1, characterized in that the non-bonding thin-film layer is formed to provide an intersection.

According to this invention, by forming plural, say, two non-bonding thin-film layers to intersect each other, a microchannel chip that can be used in electrophoresis is easily obtained.

As a means for solving the first-mentioned object, the invention provides a microchannel chip characterized in that the non-bonding thin-film layer is formed on the bonding side of the lower substrate whereas the port is formed in the upper substrate.

According to this invention, the port and the non-bonding thin-film layer can be formed separately.

As a means for solving the first-mentioned object, the non-bonding thin-film layer is formed on the bonding side of the upper substrate and that the port is formed in the upper substrate.

According to this invention, the port and the non-bonding thin-film layer can be formed on only one substrate, so the other substrate needs only to be attached to that one substrate.

As a means for solving the first-mentioned object, the invention recited above provides a microchannel chip characterized in that the non-bonding thin-film layer is formed on both the bonding side of the upper substrate and the bonding side of the lower substrate whereas the port is formed in the upper substrate.

According to this invention, the lower substrate and the upper substrate can be rendered more positive in their non-bonding properties and the area that corresponds to the non-bonding thin-film layer becomes all the more easy to inflate upon application of a positive pressure.

As a means for solving the first-mentioned object, a material spotted layer is further formed in a position that corresponds to the non-bonding thin-film layer

According to this invention, materials that are readily decomposed or invaded by moisture, oxygen, microorganisms and the like in the air, as well as materials that are readily moved by impact or environmental pressure can be stably sealed or shielded, or safely preserved or protected from those external effects until just before use.

As a means for solving the first-mentioned object, the invention provides a microchannel chip, wherein the material spotted layer is formed in a position that corresponds to the non-bonding thin-film layer and on the substrate where the non-bonding thin-film layer is not provided.

According to this invention, the material spotted layer and the non-bonding thin-film layer can be formed separately.

As a means for solving the first-mentioned object, the invention recited provides a microchannel chip, wherein the material spotted layer is formed on the non-bonding thin-film layer.

According to this invention, there can be dealt with the case where the material spotted layer cannot be formed on the substrate where the non-bonding thin-film layer is not provided.

As a means for solving the first-mentioned object, the material spotted layer is formed of at least one material selected from the group consisting of chemical reaction reagents, solutes, salts, saccharides, antigens, antibodies, physiologically active substances, endocrine disrupters, sugar chains, glycoproteins, peptides, proteins, amino acids, DNAs, RNAs, microorganisms, yeasts, fungi, spores, fragmentary plant tissues, fragmentary animal tissues, drugs, glass particles, resin particles, magnetic particles, metal particles, polymers, swollen gels, and solidified gels.

According to this invention, both non-solid and solid materials can be used as materials to form the material spotted layer.

As a means for solving the first-mentioned object, the invention provides a microchannel chip, characterized in that the upper substrate is made of polydimethyl siloxane (PDMS) whereas the lower substrate is made of polydimethyl siloxane (PDMS) or glass.

According to this invention, the upper substrate and the lower substrate can be permanently bonded together without using an adhesive.

As a means for solving the second-mentioned object, the invention provides a process for producing the microchannel chip by applying the non-bonding thin-film layer to the bonding side of at least one of the two substrates through a mask having a desired through-pattern by either one of commonly employed chemical thin-film forming methods.

According to this invention, the non-bonding thin-film layer duplicating the mask pattern can be readily formed on the bonding surface of at least one of the two substrates by commonly employed chemical thin-film forming methods. The microchannel chip can be produced not only at low cost but also in high yield, as compared with the conventional method of using a mold.

As a means for solving the second-mentioned object, the invention provides a process for producing the microchannel chip by applying the non-bonding thin-film layer to the bonding side of at least one of the two substrates through a mask having a desired through-pattern by spraying a coating agent.

According to this invention, the non-bonding thin-film layer duplicating the mask pattern can be formed extremely readily on the bonding side of at least one of the two substrates without using any special apparatus. The microchannel chip can be produced not only at low cost but also in high yield, as compared with the conventional method of using a mold.

As a means for solving the second-mentioned object, the invention recited in claim 14 provides a process for producing the microchannel chip according to any one of claims 1 to 11, characterized in that the non-bonding thin-film layer is printed on the bonding side of at least one of the two substrates.

According to this invention, the non-bonding thin-film layer is formed by printing, so the microchannel chip can be produced not only at a much lower cost but also in a far higher yield, as compared with the conventional method of using a mold.

According to the present invention, functions comparable to those of the conventional microchannels can be exhibited by simply forming the non-bonding thin-film layer on one of the two substrates and then attaching the two substrates to each other; what is more, the comparable functions can be attained without providing a fluid control element such as a micro-valve. As a result, compared to the conventional case of producing microchannel chips and micro-valves by the lithographic technology, microchannel chips can not only be manufactured extremely readily but they can also be provided at a very low cost.

Compared to the microchannel chip having the conventional microchannels, another beneficiary effect of the micro-channel chip having the non-bonding thin-film layer of the present invention is that while the conventional micro-channels are prone to contain air bubbles when a liquid is fed through them; on the other hand, in the case of the non-bonding thin-film layer of the present invention, no gap that functions as a microchannel will form unless it is inflated by applying a positive pressure and, hence, there is little likelihood for the entrance of air bubbles during the feeding of a liquid. If air bubbles are allowed to be present in the microchannel, not only is it difficult to feed the liquid in the subsequent stage but it has also been very difficult to remove the air bubbles. Hence, with the microchannel chip having the conventional microchannels, it has been necessary to feed the liquid with utmost care being taken to prevent the entrance of air bubbles, thus causing a waste of time in the liquid feeding operation. With the microchannel chip of the present invention, there is no need to waste manpower in the liquid feeding operation.

### BRIEF DESCRIPTION OF THE DRAWINGS

FIG. 1A is an outline plan view showing an example of the microchannel chip according to the present invention.

FIG. 1B is a sectional view taken through FIG. 1A along line 1B-1B.

FIG. 2A is a partial outline sectional view showing an exemplary mode of using the microchannel chip of the present invention.

FIG. 2B is a partial outline sectional view showing how the microchannel chip of FIG. 2A has slightly inflated only in the area that corresponds to a non-bonding thin-film layer 11 to thereby create a gap 18 that can function as a microchannel.

FIG. 3 is a flowchart showing an exemplary process for producing a microchannel chip according to an embodiment of the present invention.

FIG. 4A is a flowchart showing the first half of an exemplary process for producing a microchannel chip according to another embodiment of the present invention.

FIG. 4B is a flowchart showing the second half of the process for producing the microchannel chip according to the embodiment shown in FIG. 4A.

FIG. 5A is an outline plan view showing another embodiment of the microchannel chip according to the present invention.

FIG. 5B is a sectional view taken through FIG. 5A along line 5B-5B.

FIG. 6A is an outline plan view showing yet another embodiment of the microchannel chip according to the present invention.

FIG. 6B is a sectional view taken through FIG. 6A along line 6B-6B.

FIG. 6C is a partial outline sectional view showing an exemplary mode of using the microchannel chip of the present invention shown in FIG. 6B.

FIG. 7A is an outline plan view showing still another embodiment of the microchannel chip according to the present invention.

FIG. 7B is a partial outline sectional view showing how the microchannel chip 1D of FIG. 7A has slightly inflated only in the area that corresponds to the non-bonding thin-film layer 11 to thereby create a gap 18, whereupon hollow channels 104 on opposite sides of the non-bonding thin-film layer 11 come to communicate with each other.

FIG. 8A is an outline plan view showing an example of the conventional microchannel chip.

FIG. 8B is a sectional view taken through FIG. 8A along line 8B-8B.

FIG. 9 is a flowchart showing an example of the conventional process for producing the microchannel chip shown in FIGS. 8A and 8B.

### DETAILED DESCRIPTION OF THE INVENTION

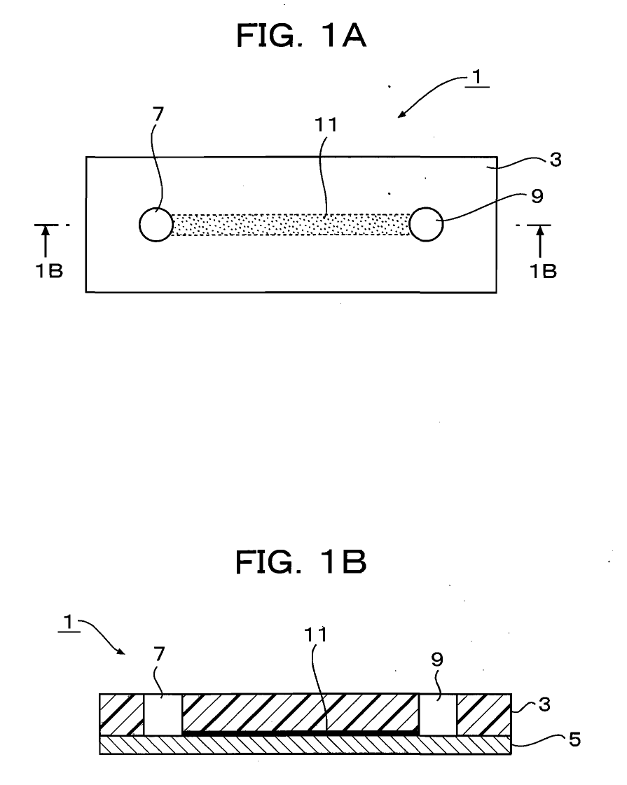

FIG. 1A is an outline plan view showing an example of the microchannel chip according to the present invention, and FIG. 1B is a sectional view taken through FIG. 1A along line 1B-1B. Like the conventional microchannel chip, the microchannel chip according to the present invention comprises an upper substrate 3 and a lower substrate 5. The upper substrate 3 has ports 7 and 9 provided in it that should serve as an inlet and an outlet for a medium such as a liquid or gas. The upper substrate 3 and the lower substrate 5 are bonded together, except in areas that correspond to a non-bonding thin-film layer 11 and the ports 7 and 9. As will be explained below in detail, the non-bonding thin-film layer 11 is an area that should serve as a microchannel in the conventional microchannel chip. However, since the ports 7 and 9 are usually interrupted by the non-bonding thin-film layer 11, a medium such as a liquid or gas cannot be transferred from one port to the other.

The non-bonding thin-film layer 11 may be exemplified by the following that can be formed by known conventional chemical thin-film forming techniques: electrode film, dielectric protective film, semiconductor film, transparent conductive film, fluorescent film, superconductive film, dielectric film, solar cell film, anti-reflective film, wear-resistant film, optical interfering film, reflective film, antistatic film, conductive film, anti-fouling film, hard coating film, barrier film, electromagnetic wave shielding film, IR shield film, UV absorption film, lubricating film, shape-memory film, magnetic recording film, light-emitting device film, biocompatible film, corrosion-resistant film, catalytic film, gas sensor film, etc.

An example of the chemical thin-film forming techniques that can be used to form the non-bonding thin-film layer 11 is by forming thin films with an apparatus for plasma discharge treatment that preferably uses an organofluorine compound or a metal compound as the reactive gas.

Exemplary organofluorine compounds that can be used in this thin-film forming method include: fluorocarbon compounds such as fluoromethanes (e.g., fluoromethane, difluoromethane, trifluoromethane, and tetrafluoromethane), fluoroethane (e.g., hexafluoroethane), 1,1,2,2-tetrafluoroethylene, 1,1,1,2,3,3,-hexafluoropropane, hexafluoropropane, and 6-fluoropropylene; fluorohydrocarbon compounds such as 1,1-difluoroethylene, 1,1,1,2-tetrafluoroethane, and 1,1,2,2,3-pentafluoropropane; fluorochlorohydrocarbon compounds such as difluorodichloromethane and trifluorochloromethane; fluoroalcohols such as 1,1,1,3,3,3-hexafluoro-2-propanol, 1,3-difluoro-2-propanol, and perfluorobutanol; fluorocarboxylate esters such as vinyl trifluoroacetate and 1,1,1-trifluoroacetate; and ketone fluorides such as acetyl fluoride, hexafluoroacetone, and 1,1,1-trifluoroacetone.

Exemplary metal compounds that can be used in this thin-film forming method include elementary or alloyed metal compounds or organometallic compounds of Al, As, Au, B, Bi, Ca, Cd, Cr, Co, Cu, Fe, Ga, Ge, Hg, In, Li, Mg, Mn, Mo, Na, Ni, Pb, Pt, Rh, Sb, Se, Si, Sn, Ti, V, W, Y, Zn, Zr, etc.

Another chemical film forming technique that may be employed is the formation of a dense film by the sol-gel method and examples of the metal compounds that are preferred as the sol-gel include elementary or alloyed metal compounds or organometallic compounds of Al, As, Au, B, Bi, Ca, Cd, Cr, Co, Cu, Fe, Ga, Ge, Hg, In, Li, Mg, Mn, Mo, Na, Ni, Pb, Pt, Rh, Sb, Se, Si, Sn, Ti, V, W, Y, Zn, Zr, etc. The non-bonding thin-film layer 11 may also be formed by methods other than those mentioned above. For instance, the non-bonding thin-film layer 11 may be formed on the upper surface of the lower substrate 5 by printing. For printing, a variety of known and conventional printing methods may be adopted, including roll printing, silk printing, pattern printing, decalcomania, electrostatic duplication, and the like. When the non-bonding thin-film layer 11 is to be formed by printing techniques, various materials can advantageously be used to form the non-bonding thin-film layer 11 and they include: fine metal particles [for example, the fine particles of elementary metals as selected from among Al, As, Au, B, Bi, Ca, Cd, Cr, Co, Cu, Fe, Ga, Ge, Hg, In, Li, Mg, Mn, Mo, Na, Ni, Pb, Pt, Rh, Sb, Se, Si, Sn, Ti, V, W, Y, Zn, Zr, etc. or alloys of two or more species thereof or the fine particles of oxides of these elementary metals or alloys thereof (e.g. fine ITO particles), and the fine particles of organometallic compounds of these metals], conductive ink, insulated ink, fine carbon particles, silanizing agent, parylene, coatings, pigments, dyes, water-based dye ink, water-based pigment ink, oil-based dye ink, oil-based pigment ink, solvent-based ink, solid ink, gel ink, polymer ink, and the like.

Alternatively, the non-bonding thin-film layer 11 can also be formed by spray coating. For instance, a coating agent may be sprayed from above a mask having a specified channel pattern and subsequently dried to form the non-bonding thin-film layer 11 on the upper surface of the lower substrate 5. For example, a material capable of forming a coating, as exemplified by an electrode coating, dielectric protective coating, semiconductor coating, conductive coating, fluorescent coating, superconductive coating, dielectric coating, anti-reflective coating, wear-resistant coating, optical interfering coating, reflective coating, antistatic coating, anti-fouling coating, hard coated coating, barrier coating, electromagnetic wave shielding coating, IR shield coating, UV absorption coating, lubricating coating, light-emitting device coating, biocompatible coating, corrosion-resistant coating, catalytic coating, a metal coating, a glass coating, an applied coating, a water-repellent coating, a hydrophilic coating, a resin coating, a rubber coating, a synthetic fiber coating, a synthetic resin coating, a phospholipid coating, a coating made of a bio-derived substance, a bio-substance anti-bonding coating, a lipid coating, an oil coating, a silane compound coating, a silazane compound coating or a sticky coating, may be dissolved or suspended in a suitable solvent, with the resulting solution or suspension being sprayed as a coating agent.

The film thickness of the non-bonding thin-film layer 11 varies with the thin-film forming method used but its is preferably within the range from 10 nm to 300 μm. If the thickness of the non-bonding thin-film layer 11 is less than 10 nm, the non-bonding thin-film layer 11 will not be formed uniformly but both bonding and non-bonding sites will be scattered about as islands and the non-bonding thin-film layer 11 finds difficulty functioning to provide a micro-channel. On the other hand, if the thickness of the non-bonding thin-film layer 11 is greater than 300 μm, not only is the non-bonding effect saturated but due the excessive thickness of the non-bonding thin-film layer 11, the upper substrate 3 also comes apart at the border of bonding to the non-bonding thin-film layer 11 and fails to be bonded effectively. This causes undesirable inconveniences such as the failure to maintain the exact width of the non-bonding thin-film layer 11. If the chemical thin-film forming method is employed, the thickness of the non-bonding thin-film layer 11 generally ranges from 10 nm to 10 μm, preferably from 30 nm to 5 μm, more preferably from 50 nm to 3 μm. If the spray coating method is employed, the thickness of the non-bonding thin-film layer 11 generally ranges from 50 nm to 300 μm, preferably from 80 nm to 200 μm, more preferably from 100 nm to 100 μm. If the printing method is employed, the thickness of the non-bonding thin-film layer 11 generally ranges from 500 nm to 100 μm, preferably from 800 nm to 80 μm, more preferably from 1 μm to 50 μm.

The width of the non-bonding thin-film layer 11 may be generally the same as or greater or even smaller than the width of microchannels in the conventional microchannel chip. Generally, the non-bonding thin-film layer 11 has a width ranging from about 10 μm to about 3000 μm. If the width of the non-bonding thin-film layer 11 is less than 10 μm, so high a pressure must be exerted to inflate the non-bonding area for creating a microchannel that the microchannel chip 1 itself might be destroyed. On the other hand, if the width of the non-bonding thin-film layer 11 exceeds 3000 μm, the channel that is formed by inflating over a width greater than 3000 μm will be saturated with an unduly large amount of substance although the microchannel chip is inherently intended to transport and control very small amounts of liquid or gas and perform chemical reaction, synthesis, purification, extraction, generation and/or analysis on substances. An additional undesirable inconvenience is the likelihood to impair the ability of the channel to prevent liquid deposition on its inner surfaces although this ability is one of the advantages of the channel structure obtained by inflating.

The pattern of the non-bonding thin-film layer 11 is by no means limited to the illustrated linear form. In consideration of the object and/or use, the non-bonding thin-film layer 11 in Y-shaped, L-shaped or various other patterns may be adopted. If desired, the non-bonding thin-film layer 11 having a port at both ends may be more than one in number. A plurality of such non-bonding thin-film layers 11 with ports may be arranged in any pattern such as a parallel or crossed array. The crossed array is useful in the conventional cross-injectable electrophoretic chip. Furthermore, in addition to the linear portion, the non-bonding thin-film layer 11 may also have an enlarged region in any planar shape, such as a circular, an elliptical, a rectangular, or a polygonal shape. The enlarged region can also be used as a reaction compartment.

The upper substrate 3 of the microchannel chip 1 according to the present invention does not necessarily have elasticity and/or flexibility but it is generally preferred that it be made of a polymer or an elastomer. If the upper substrate 3 is not formed of an elastic and/or flexible material, it becomes either impossible or difficult to ensure that the part of the upper substrate 3 which corresponds to the non-bonding thin-film layer 11 is sufficiently deformed to create a microchannel of the type found in the conventional microchannel chip. Hence, preferred materials of which the upper substrate 3 can be formed include not only silicone rubbers such as polydimethyl siloxane (PDMS) but also the following: nitrile rubber, hydrogenated nitrile rubber, fluorinated rubber, ethylene-propylene rubber, chloroprene rubber, acrylic rubber, butyl rubber, urethane rubber, chlorosulfonated polyethylene rubber, epichlorohydrin rubber, natural rubber, isoprene rubber, styrene-butadiene rubber, butadiene rubber, polysulfide rubber, norbornene rubber, and thermoplastic elastomers. Silicone rubbers such as polydimethyl siloxane (PDMS) are particularly preferred.

The thickness of the upper substrate 3 is within the range from 10 μm to 5 mm. If the thickness of the upper substrate 3 is less than 10 μm, even a low pressure is sufficient for creating a microchannel by inflating that part of the upper substrate 3 which corresponds to the non-bonding thin-film layer 11 but, on the other hand, there is a high likelihood for the upper substrate 3 to rupture. If the thickness of the upper substrate 3 exceeds 5 mm, an undesirably high pressure must be exerted to create a microchannel by inflating that part of the upper substrate 3 which corresponds to the non-bonding thin-film layer 11.

The lower substrate 5 of the microchannel chip 1 according to the present invention does not necessarily have elasticity and/or flexibility but it is preferred that it can be strongly bonded to the upper substrate 3. “Strongly bonded” means such a bonding power that those bonding portions other than the non-bonding thin-film layer enable the creation of a channel structure due to deformation by inflation of the site corresponding to the non-bonding thin-film layer. Furthermore, the channel structure created from deformation by inflation of the site corresponding to the non-bonding thin-film layer is occasionally filled under pressure with a liquid, gas, vapor, or a polymer or gel-like substance that are moved under pressure or by squeezing and there is required a bonding strength that can withstand such pressure application or squeezing. Suppose the case where the upper substrate 3 is made of polydimethyl siloxane (PDMS); if the lower substrate 5 is made of PDMS or glass, the upper substrate 3 and the lower substrate 5 can be strongly bonded to each other. This phenomenon is generally called “permanent bonding.” Permanent bonding refers to such a property that the upper substrate and the lower substrate, both made of PDMS, can be strongly bonded to each other without using an adhesive but by just performing a certain kind of surface modification; this property contributes to exhibiting an effective seal on microstructures such as microchannels and/or ports. In the permanent bonding of PDMS substrates, their mating surfaces are subjected to an appropriate treatment of surface modification and then the two substrates are superposed, with their mating surfaces being placed in intimate contact with each other, and the assembly is left to stand for a certain period of time, whereupon the two substrates can be easily bonded together. In other words, those parts of the substrates which correspond to the non-bonding thin-film layer 11 are not permanently bonded, so pressure or other external force may be applied to inflate those portions in a balloon-like shape for creating a micro-channel and a micro-valve. Since the portions other than those inflated are permanently bonded, the liquid or gas that is passed through the inflated portions will not leak to any other sites.

As long as permanent bonding to the upper PDMS substrate 3 is possible, it is of course possible to use the lower substrate 5 that is made of materials other than PDMS or glass. Examples of such lower substrate include cellulose ester substrates, polyester substrates, polycarbonate substrates, polystyrene substrates, polyolefin substrates, etc.; specific examples include poly(ethylene terephthalate), poly(ethylene naphthalate), polyethylene, polypropylene, cellophane, cellulose diacetate, cellulose acetate butyrate, cellulose acetate propionate, cellulose acetate phthalate, cellulose triacetate, cellulose nitrate, poly(vinylidene chloride), poly(vinyl alcohol), ethylene-vinyl alcohol, polycarbonate, norbornene resin, poly(methylpentene), polyetherketone, polyimide, polyethersulfone, poly(etherketone imide), polyamide, fluororesin, nylon, poly(methyl methacrylate), acrylics, polyallylate, etc. Other materials that can be used to form the lower substrate 5 include poly(lactic acid) resins, poly(butylene succinate), nitrile rubber, hydrogenated nitrile rubber, fluorinated rubber, ethylene-propylene rubber, chloroprene rubber, acrylic rubber, butyl rubber, urethane rubber, chlorosulfonated polyethylene rubber, epichlorohydrin rubber, natural rubber, isoprene rubber, styrene-butadiene rubber, butadiene rubber, polysulfide rubber, norbornene rubber, and thermoplastic elastomers. These materials can be used either alone or in suitable admixture.

If these materials are not capable of permanent bonding by themselves, their surfaces to be bonded to the upper substrate 3 are subjected to such a surface treatment that they can be permanently bonded. Agents that can be used in this surface treatment include silicone compounds and titanium compounds and specific examples include: organosilicon compounds including alkyl silanes such as dimethylsilane, tetramethylsilane, and tetraethylsilane, as well as silicon alkoxysilanes such as tetramethoxysilane, tetraethoxysilane, tetrapropoxysilane, dimethyldiethoxysilane, methyltrimethoxysilane, and ethyltriethoxysilane; silicon hydride compounds such as monosilane and disilane; silicon halide compounds such as dichlorosilane, trichlorosilane, and tetrachlorosilane; silazanes such as hexamethyldisilazane; and silicon compounds having functional groups introduced therein, as exemplified by vinyl, epoxy, styryl, methacryloxy, acryloxy, amino, ureido, chloropropyl, mercapto, sulfide, and isocyanate. These agents for surface treatment can be used alone but two or more kinds of them may be used in suitable admixture.

It is generally preferred that the thickness of the lower substrate 5 is within the range from 300 μm to 10 mm. If the thickness of the lower substrate 5 is less than 300 μm, it becomes difficult to maintain the overall mechanical strength of the microchannel chip 1. If, on the other hand, the thickness of the lower substrate 5 exceeds 10 mm, the mechanical strength required of the microchannel chip 1 is saturated and only diseconomy results.

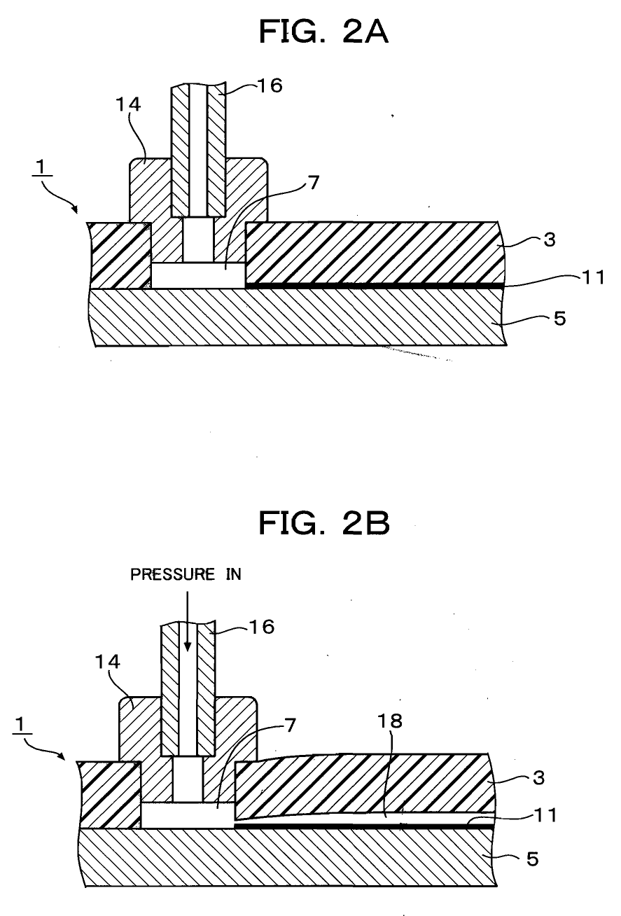

FIG. 2 is a set of partial outline sectional views showing an exemplary mode of using the microchannel chip 1 of the present invention. As shown in FIG. 2A, the microchannel chip 1 of the present invention has an adapter 14 provided in the opening of the port 7 which should help introduce a liquid or gas and a feed tube 16 is connected to this adapter 14. Needless to say, the shape of the adapter 14 is not limited to the illustrated example. Instead of a shape that permits partial insertion into the port, it may assume a shape that enables it to be directly secured to the upper substrate 3. Alternatively, the adapter 14 may be dispensed with and the feed tube 16 may be directly connected to each port. The adapter 14 may be formed of PDMS which can permanently bond to the upper substrate 3 which is made of PDMS but other materials can of course be employed. If the adapter 14 is not made of PDMS, a suitable adhesive may be employed to secure it to the upper substrate 3. The feed tube 16 is formed of a flexible material. For instance, a TEFLON (registered trademark) tube is preferred. The feed tube 16 can be secured to the adapter 14 by using a suitable adhesive. Although not shown, the other end of the feed tube 16 is connected to a suitable liquid feed supply means and/or pressure applying means (e.g. a micro-pump or syringe). If a liquid of interest has been injected into the port 7, a gas (e.g. air) is forced through the feed tube 16 at high pressure (say, 10 kPa to 100 kPa). Alternatively, a liquid of interest is injected into the port 7 with a positive pressure being simultaneously applied. Then, as shown in FIG. 2B, only that part of the upper substrate 3 which corresponds to the non-bonding thin-film layer 11 is slightly inflated to create a gap 18 that can function as a microchannel, whereupon the liquid and/or gas within the port 7 can be transferred to the port 9. If the outer surface of the topside of the upper substrate 3 that corresponds to the non-bonding thin-film layer 11 is pressed with a finger or something like that, the gap 18 created by inflating can be readily closed. Therefore, with the microchannel chip 1 of the present invention, no special constituent element like the conventional micro-valve need be provided and yet an operational effect comparable to that obtained by the micro-valve can be exhibited.

In the embodiment shown in FIG. 1, the two ends of the non-bonding thin-film layer 11 are connected to the ports 7 and 9 that are open to the atmosphere, but it may be connected to only one port. As long as at least one end of the non-bonding thin-film layer 11 is connected to a port open to the atmosphere, one may apply a positive pressure via the port open to the atmosphere, whereupon the part that corresponds to the non-bonding thin-film layer 11 is inflated to create a gap that can function as a microchannel. This is the same principle as that for inflating a balloon. As a result, a liquid and/or a gas can be forced through one port into the gap created by inflating. If both ends of the non-bonding thin-film layer are connected to the port open to the atmosphere, the liquid and/or gas can be transferred from one port to the other.

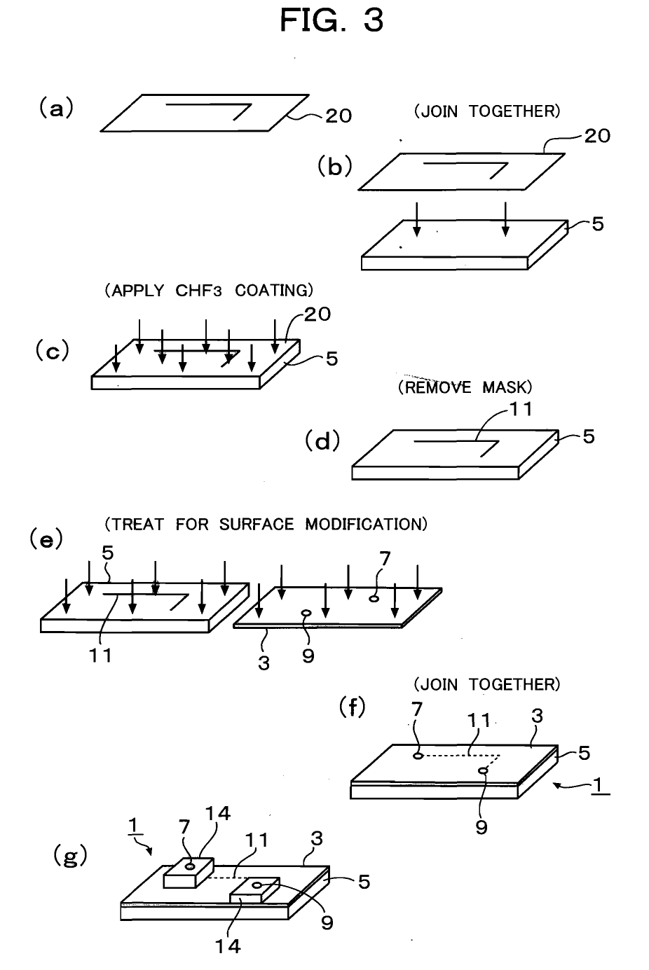

FIG. 3 is a flowchart showing an exemplary process for producing the microchannel chip 1 of the present invention. To begin with, in step (a), there is provided a mask 20 having a pattern of a specified channel design formed on it. The mask can be formed of a synthetic resin film (e.g., PET film or a vinyl chloride film) or a metal foil or the like in a thickness of about 0.01 mm to about 1 mm. Hence, a mask having a desired through-pattern can be fabricated by punching a film or metal foil with a die, cutting with a knife, or electrical discharge machining with a laser or the like, or machining with a cutter. In step (b), the mask 20 is attached to the upper surface of a base material (e.g., PDMS) that should provide the lower substrate 5, either by utilizing such a phenomenon as adsorption or by bonding. In step (c), the assembly is treated with a reactive ion etching system (RIE) in the presence of trifluoromethane (CHF₃), whereby the lower substrate 5 is coated with a pattern of trifluoromethane (CHF₃) that duplicates the channel design. In step (d), the mask 20 is stripped, leaving on the upper surface of the lower substrate 5 a non-bonding thin-film layer 11 that is made of trifluoromethane (CHF₃) in a pattern that duplicates the channel design. Alternatively, a waterproof spray of a silicon acrylic resin-based water repellent that is generally available on the market may be trickled or otherwise applied from above the mask 20 to coat the lower substrate 5 with a pattern of silicon acrylic resin-based water repellent that duplicates the channel design, thereby forming a non-bonding thin-film layer 11 made of the silicon acrylic resin-based water repellent. In step (e), the upper surface of the lower substrate 5 where the non-bonding thin-film layer 11 is present, as well as the lower side of the upper substrate 3 in which through-holes for the ports 7 and 9 have been made are treated for surface modification. Exemplary methods of treatment for surface modification are treatment with an oxygen plasma, treatment by irradiation with excimer UV light, and the like. Treatment with an oxygen plasma can be implemented with a reactive ion etching (RIE) apparatus. Treatment by irradiation with excimer UV light features a lower treatment cost since it can be implemented in an air atmosphere under atmospheric pressure using a dielectric barrier discharge lamp. Subsequently, in step (f), the sides that have been treated for surface modification are attached to each other so that the upper substrate 3 and the lower substrate 5 are permanently bonded. If a feed tube is to be directly connected to each port, the microchannel chip 1 of the present invention is completed at this stage. However, if desired, the process may proceed to the final step (g), where an adapter 14 for assisting in connection to the feed tube is secured to the respective sites of ports 7 and 9 to obtain the microchannel chip 1 of the present invention.

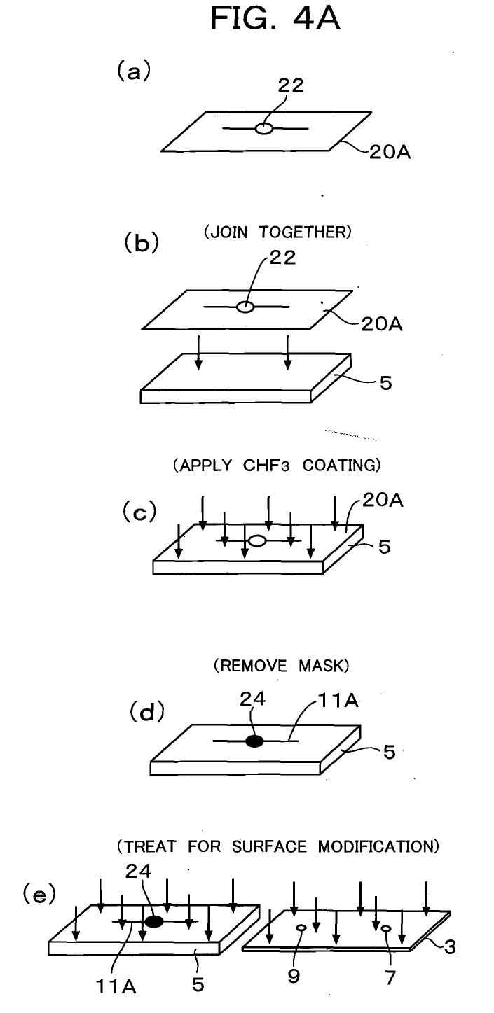

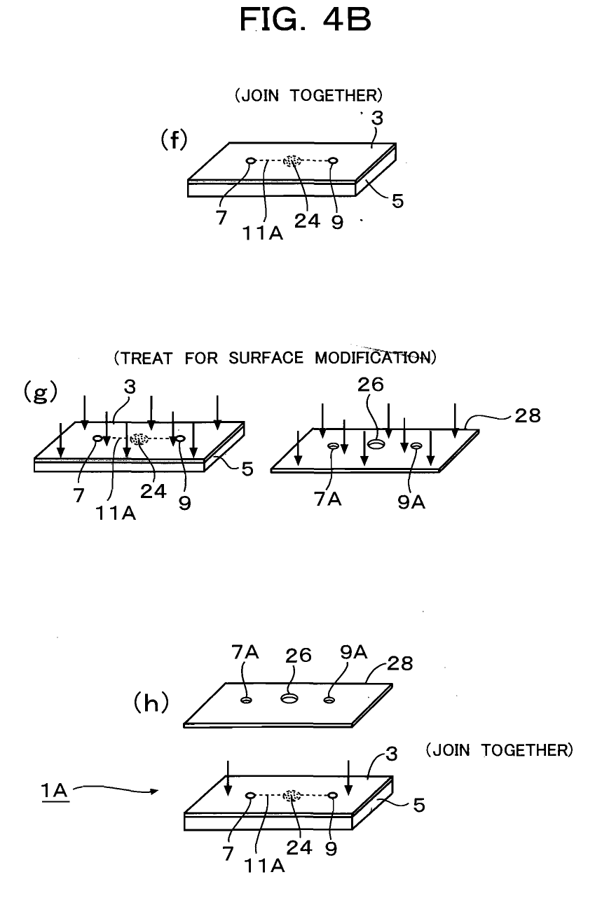

FIGS. 4A and 4B are flowcharts showing an exemplary process for producing the microchannel chip 1A according to another embodiment. The production process depicted in FIGS. 4A and 4B is again basically the same as the production process shown in FIG. 3. To begin with, in step (a), there is provided a mask 20A having a pattern of a specified channel design. The mask 20A differs from the mask 20 of FIG. 3 in that it has a through-hole 22 for forming a liquid reservoir site. In step (b), the mask 20A is attached to the upper surface of a base material (e.g., PDMS) that should provide the lower substrate 5, either by utilizing such a phenomenon as adsorption or by bonding. In step (c), the assembly is treated with a reactive ion etching system (RIE) in the presence of trifluoromethane (CHF₃), whereby the lower substrate 5 is coated with a pattern of trifluoromethane (CHF₃) that duplicates the channel design. In step (d), the mask 20A is stripped, leaving on the upper surface of the lower substrate 5 a non-bonding thin-film layer 11A that is made of trifluoromethane (CHF₃) in a pattern that duplicates the channel design. Alternatively, a waterproof spray of a silicon acrylic resin-based water repellent that is generally available on the market may be trickled or otherwise applied from above the mask 20A to coat the lower substrate 5 with a pattern of silicon acrylic resin-based water repellent that duplicates the channel design, thereby forming a non-bonding thin-film layer 11A made of the silicon acrylic resin-based water repellent. The non-bonding thin-film layer 11A differs from the non-bonding thin-film layer 11 of FIG. 3 in that it has an enlarged region 24 that should serve as a liquid reservoir site. In step (e), the upper surface of the lower substrate 5 where the non-bonding thin-film layer 11A is present, as well as the lower side of the upper substrate 3 in which through-holes for the ports 7 and 9 have been made are treated for surface modification. Subsequently, in step (f), the sides that have been treated for surface modification are attached to each other so that the upper substrate 3 and the lower substrate 5 are permanently bonded. In step (g), the lower side of a silicone rubber sheet 28 that has a specified sufficient thickness (e.g., 1 mm) that it can also function as ports and which have formed therein two port sites and a through-hole 26 of the same shape (e.g., a circle with a diameter of 5 mm) as that of the liquid reservoir site 24, as well as the upper side of the assembly obtained in the aforementioned step (f) are treated for surface modification. The diameter of the through-hole 26 is preferably equal to or greater than the diameter of the liquid reservoir site 24. Finally, in step (h), the silicone rubber sheet 28 and the upper substrate 3 are attached to each other by permanent bonding, with the through-holes 7A and 9A in the former being placed in registry with the ports 7 and 9 in the latter, so as to complete the intended microchannel chip 1A. Although not shown, if desired, an adapter 14 for assisting in connection to the feed tube may be secured to the through-holes 7A and 9A in the silicone rubber 28. Treating the silicone rubber sheet 28 for surface modification is not an essential requirement of the present invention. The silicone rubber sheet 28 need not be treated for surface modification but it may simply be self-adsorbed to the upper substrate 3. Needless to say, the foregoing description concerning the method of forming the non-bonding thin-film layer 11, its thickness, feature size, pattern, and the like is equally applicable to the non-bonding thin-film layer 11A. Thus, the description concerning the method of forming the non-bonding thin-film layer 11A, its thickness, feature size, pattern, and the like is simply redundant and hence is omitted.

In each of the production methods described above, the non-bonding thin-film layer 11 or 11A may be provided on the upper substrate, rather than on the lower substrate. In this case, all tiny constituent elements such as the ports and the non-bonding thin-film layer are provided on the upper substrate, so there is no need to apply microfabrication to the lower substrate and the production of the microchannel chip can be further simplified.

In yet another embodiment, the non-bonding thin-film layer 11 or 11A may be provided on both the lower substrate and the upper substrate. In this case, the lower substrate and the upper substrate can be rendered more positive in their non-bonding properties and the area that corresponds to the non-bonding thin-film layer 11 or 1A becomes all the more easy to inflate upon application of a positive pressure.

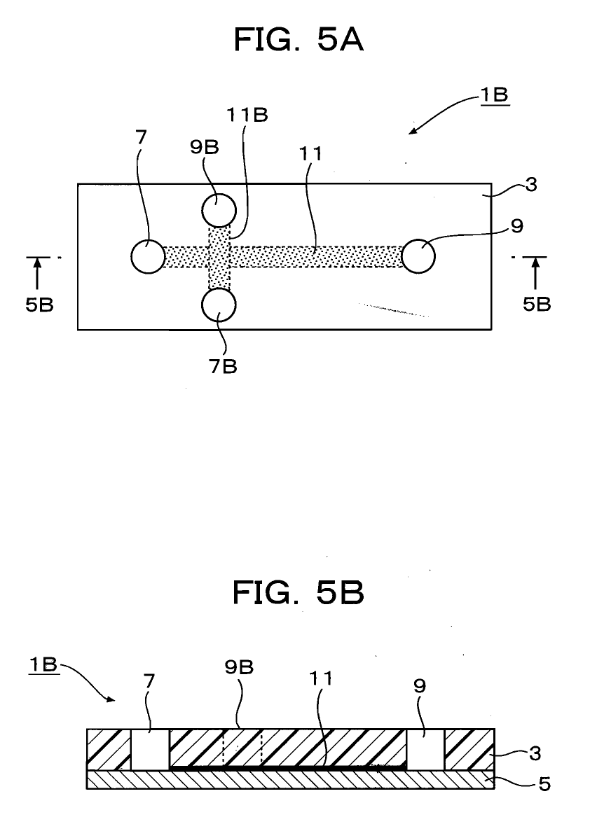

FIGS. 5A and 5B are a plan view and a sectional view that show yet another embodiment of the microchannel chip according to the present invention. As shown, a main non-bonding thin-film layer 11 is crossed by a sub-non-bonding thin-film layer 11B. Needless to say, the foregoing description concerning the method of forming the non-bonding thin-film layer 11, its thickness, feature size, pattern, and the like is equally applicable to the non-bonding thin-film layer 11B. The microchannel chip 1B is particularly suitable as a cross-injectable electrophoretic chip. Consider, for example, the case where there occurs the need to use the microchannel chip 1B as an electrophoretic chip; a gel electrolyte is packed from the port 9 toward the port 7, as well the ports 7B and 9B, so that the non-bonding thin-film layers 11 and 11B are inflated as described above to provide micro-electrophoretic channels and electrophoresis is then performed. After confirming that the gel electrolyte packed from the port 9 has overflowed the ports 7, as well as the ports 7B and 9B, the same gel electrolyte is also packed into the ports 7, as well as the ports 7B and 9B. Subsequently, an analyte to be electrophoresed is injected into the port 7B and electrodes are dipped into the ports 7 and 9, as well as the ports 7B and 9B. First, a voltage is applied between the electrodes at the ports 7B and 9B. In response to this voltage application, the analyte in the port 7B is migrated through the inflated channel 11B toward the port 9B. By a suitable optical detection means (not shown), the analyte is confirmed to have been migrated to the point of crossing between the inflated channel 11B and the other inflated channel 11, and voltage is now applied between the electrodes at the ports 7 and 9. As a result of this changeover in voltage application, the analyte located at the point of crossing between the inflated channels 11B and 11 is migrated toward the port 9, so a specified detecting process can be performed near at the port 9 by a suitable optical detection means (not shown). In the prior art, electrophoretic microchannel chips of the type described above have been fabricated by the complicated lithographic technology and the like but according to the present invention, they can be mass-produced at lower cost by the simple method as described above.

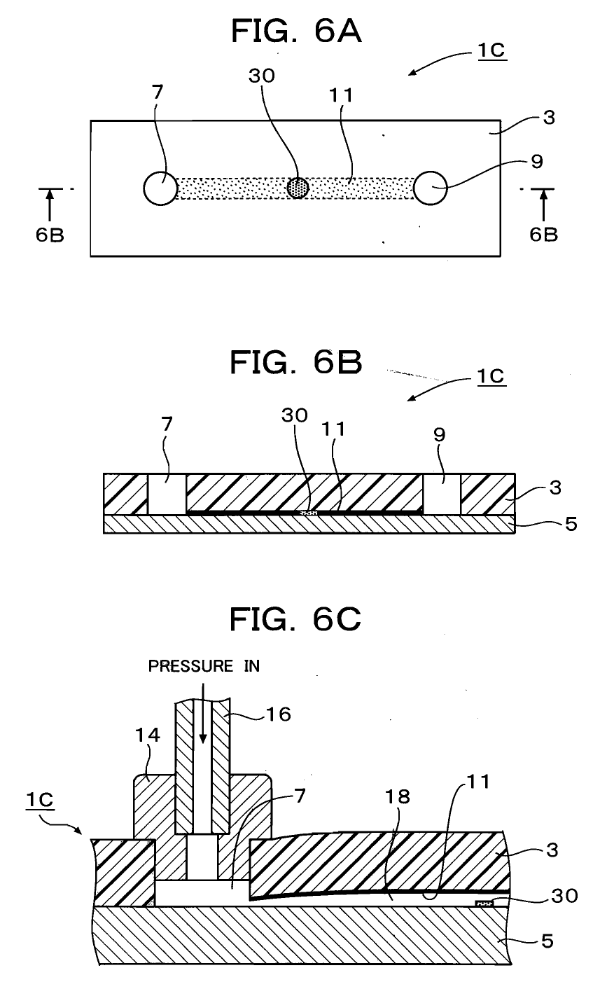

FIGS. 6A, 6B and 6C are a plan view and sectional views that show still another embodiment of the microchannel chip according to the present invention. The microchannel chip 1C according to this embodiment has a material spotted layer 30 in a position that corresponds to the non-bonding thin-film layer 11. An advantage of the microchannel chip 1C according to this embodiment is that materials that are readily decomposed or invaded by moisture, oxygen, microorganisms and the like in the air can be stably sealed or shielded, or safely preserved or protected from such moisture, oxygen, microorganisms and the like until just before use. The micro-channel chip 1C of the present invention has another advantage that, even as regards materials that are readily moved by impact or a change in environmental pressure that are exerted on chips having conventional rectangular channels and which are difficult to retain in a specified area in the channel can also be protected from wind pressure, external impact and the like and retained in the specified area until just before use.

In FIGS. 6A, 6B and 6C, the material spotted layer 30 is not limited to unity in number but a desired number of such material spotted layers may be provided. In addition, the material spotted layer 30 is not limited to the position that corresponds to the non-bonding thin-film layer 11 but it may also be provided in a position that corresponds to the enlarged region 24 which should serve as a liquid reservoir site, as shown in FIGS. 4A and 4B. The material spotted layer 30 may be formed on the lower substrate 5. However, this is not the sole embodiment that can be realized. After the non-bonding thin-film layer 11 is formed on the lower substrate 5, the material spotted layer 30 may be provided on the upper surface of the non-bonding thin-film layer 11; alternatively, it may be provided on the upper substrate 3. In the case where the lower substrate 5 is made of glass, the material spotted layer 30 may be formed on the upper surface of the glass substrate, and the non-bonding thin-film layer 11 on the lower side of the upper substrate 3.

To form the material spotted layer 30, any liquid or solid material can be used. A liquid material may be used as such, but it may first be applied and then dried to form a film. Such materials may be exemplified by chemical reaction reagents, solutes, salts, saccharides, antigens, antibodies, physiologically active substances, endocrine disrupters, sugar chains, glycoproteins, peptides, proteins, amino acids, DNAs, RNAs, microorganisms, yeasts, fungi, spores, fragmentary plant tissues, fragmentary animal tissues, drugs, glass particles, resin particles, magnetic particles, metal particles, polymers, swollen gels, and solidified gels. These materials may be used either alone or two or more kinds may be used in combination.

Therefore, the material spotted layer 30 may, for example, be oligomers for use in PCR amplification reaction (i.e., primers for use in PCR) or antigens or antibodies for use in antigen-antibody reaction or enzyme immunoassay (ELISA). ELISA may be performed by the direct adsorption procedure or the sandwich technique. In the case of direct adsorption, an antigen 30 (e.g. HIV antigen) may be adhered to a solid-phase surface of the glass substrate 5 by a suitable method such as amino coupling, surface-thiol coupling or ligand-thiol coupling. In the case of the sandwich technique, a primary antibody rather than the antigen may be bound to the solid-phase surface of the glass substrate 5. In the case of direct adsorption, a sample to be tested (e.g., serum) is injected through the port 7. Any antibody (e.g., anti-HIV antibody) in the sample will react with the antigen 30 and bind to it. Thereafter, a chromogenic reagent or the like may be injected through the port 7 so as to verify the occurrence of an antigen-antibody reaction. In the case of the sandwich technique, a solution containing the substance of interest (e.g., a protein) is injected through the port 7, whereupon the antigen in the solution binds to a primary antibody on the glass substrate 5 through the “antigen-antibody reaction.” Thereafter, an enzyme-labelled secondary antibody is injected through the port 7, enabling the substance of interest bound to the primary antibody to be determined both qualitatively and quantitatively. If the material spotted layer 30 is made of another material, say, glass particles, a sample to be tested is injected through the port 7. Any DNA in the sample is adsorbed to the glass particles. Thereafter, the glass particles may be washed with a suitable eluant, whereby only the DNA of interest can be separated.

The material spotted layer 30 may be formed by manual application or with an automatic applicator. An example of automatic applicators is a fully automatic miacroarrayer commercially available from Hitachi High-Technologies Corporation (e.g., Proteogen CM-1000). A feature of this apparatus is that in order to adhere an antigen onto the glass substrate, an immobilizing reagent called “a prolinker” is preliminarily secured to the glass substrate. With this apparatus, a standard format slide glass measuring 25.4 mm×76.2 mm is used to have a chemical reaction reagent coated automatically as spots of 100 to 300 μm in diameter on a pitch of 10 μm at a maximum density of 4900 spots/cm². In the case where the material spotted layer 30 is made of a solid material, the solid material may be suspended in a suitable solvent or the like and the suspension is applied onto the glass substrate, followed by optional drying to fix the solid material.

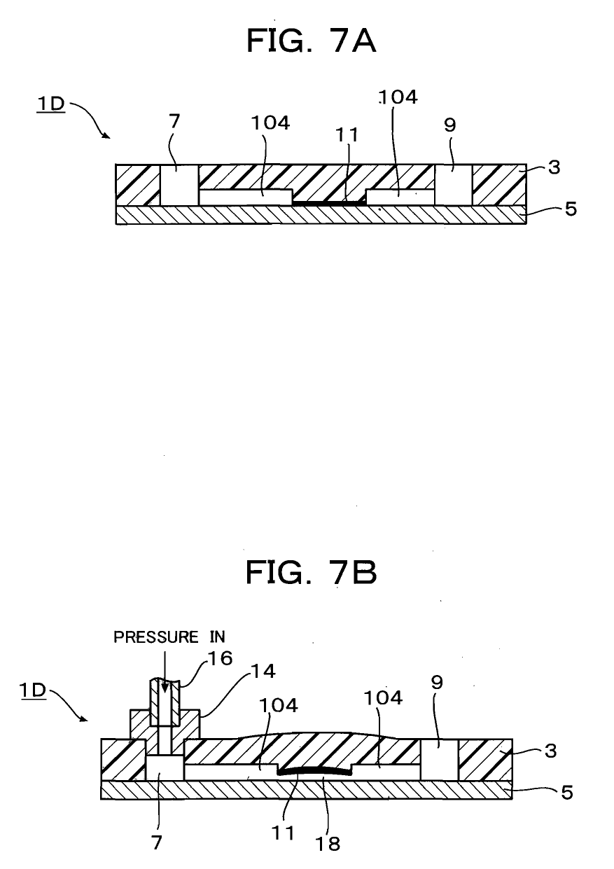

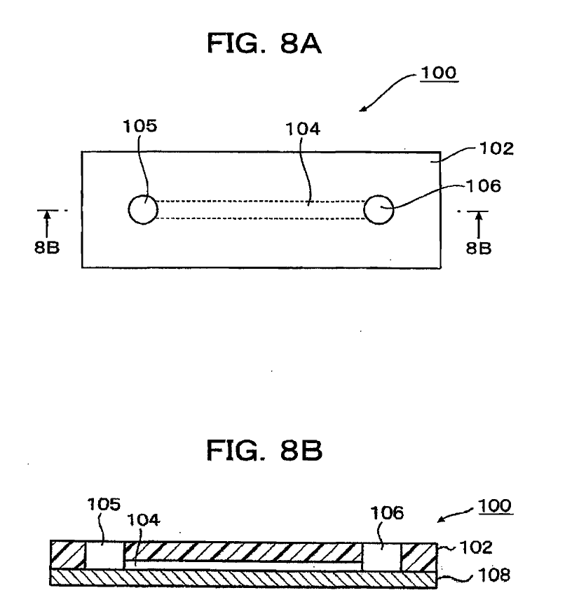

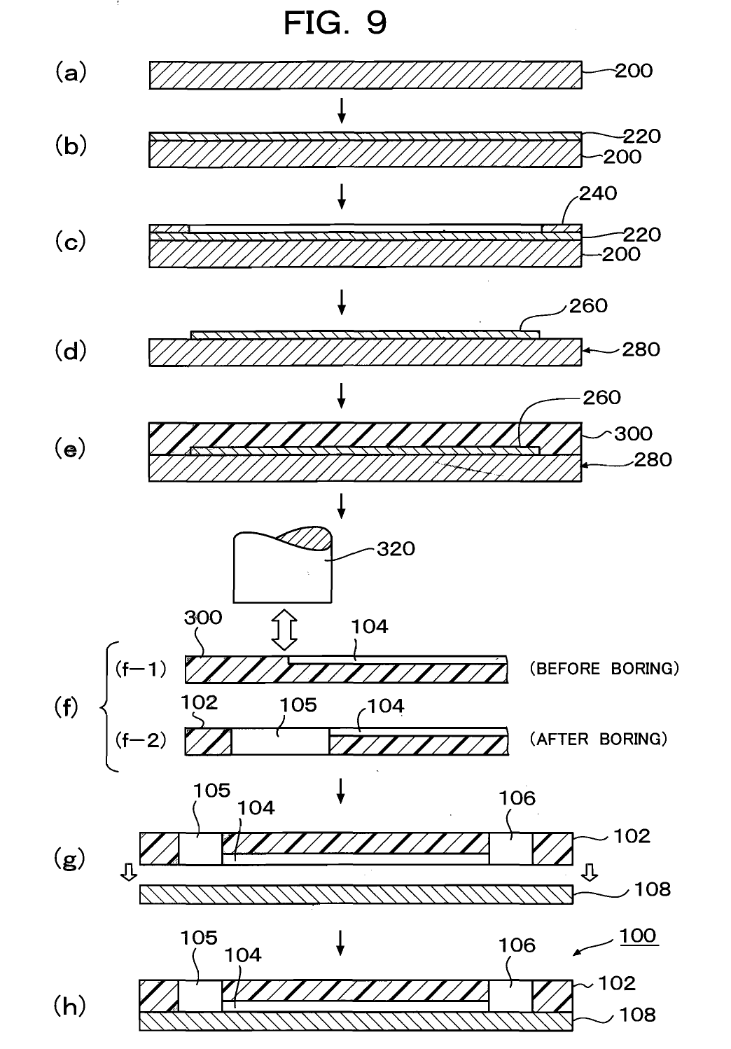

FIG. 7A is an outline sectional view showing another embodiment of the microchannel chip according to the present invention. The micro-channel chip 1D in the illustrated embodiment has a hollow microchannel 104 fabricated by the conventional mold-based lithographic technique, with a non-bonding thin-film layer 11 being provided in such a way as to interrupt or join up the hollow microchannel 104.

FIG. 7B is a partial outline sectional view showing how the microchannel chip 1D of FIG. 7A has slightly inflated only in the area where the non-bonding thin-film layer 11 is provided to create a gap 18, whereupon hollow microchannels 104 on opposite sides of the non-bonding thin-film layer 11 come to communicate with each other. An adapter 14 is provided in the opening of the port 7 which should help introduce a liquid or gas and a feed tube 16 is connected to this adapter 14. If a gas (e.g. air) is forced through the feed tube 16 at high pressure (say, 10 kPa to 100 kPa), that part of the upper substrate which corresponds to the non-bonding thin-film layer 11 is slightly inflated to create a gap 18, with the result that the hollow microchannels 104 on opposite sides of the non-bonding thin-film layer 11 come to communicate with each other. Thus, according to the embodiment under consideration, the non-bonding thin-film layer 11 can not only function as a microchannel per se but it can also fulfill the function as an on-off valve or a micro-valve between the hollow microchannels fabricated by the conventional photolithographic process.

### EXAMPLE 1

### (1) Fabrication of a Microchannel Chip

According to the flowchart shown in FIG. 3, a microchannel chip was fabricated. A mask was first provided; it was a 0.025 mm-thick PET film having a score (feature size, 400 μm) cut through in an L-shape. This mask was placed on the upper surface of a 3 mm-thick lower substrate made of PDMS and then attached to the PDMS-made lower substrate by means of self-adsorption. The assembly was housed within a reactive ion etching apparatus and a coating of trifluoromethane (CHF₃) was applied from above the mask. After the end of the CHF₃ application, the assembly was taken out of the reactive ion etching apparatus and stripped of the mask. As a result, a thin trifluoromethane (CHF₃) film with a thickness of 1 μm had been formed on the upper surface of the PDMS-made lower substrate in an L-shaped pattern. The thin patterned trifluoromethane (CHF₃) film is an area that should serve as a non-bonding thin-film layer. The upper side of the PDMS-made lower substrate having the thin patterned trifluoromethane (CHF₃) film formed on it and the lower side of a 0.1 mm-thick silicone rubber-made upper substrate having port providing through-holes with an inside diameter of 2 mm provided in specified positions were subjected to a treatment for surface modification by an oxygen plasma in the reactive ion etching apparatus. After the treatment, the lower side of the silicone rubber-made upper substrate was attached to the upper side of the PDMS-made lower substrate on which the thin patterned trifluoromethane (CHF₃) film had been formed, whereby the PDMS-made lower substrate was permanently bonded to the silicone-rubber made upper substrate. A 5 mm-thick rectangular adapter having a through-hole with an inside diameter of 2 mm was permanently bonded to each of the port sites on the silicone rubber-made upper substrate after performing the same treatment for surface modification as described above.

### (2) Liquid Feeding Test

The microchannel chip fabricated in (1) above was tested for the transferability of a liquid from one port to the other. Port 9 was charged with 1 μL of the DNA staining solution Cyber Green I and microscopically examined for the occurrence of any fluorescence. Since there was no DNA available at that time, no fluorescence was observed. Port 7 was charged with 10 μL of a solution of human genome (DNA) in TE and air pressure (positive pressure) was applied to the solution in port 7 by means of a syringe connected to the through-hole in the adapter. The pressure in the port 7 was gradually increased and at the point in time when it exceeded 50 kPa, the non-bonding area that was made of the thin patterned trifluoromethane (CHF₃) film inflated to create a gap that should serve as a microchannel, through which the solution in the port 7 was transferred toward the port 9, where the DNA solution mixed with the fluorescent reagent. Examination under a fluorescence microscope showed the emission of fluorescence from the fluorescent reagent that had intercalated into the DNA. This demonstrated that the non-bonding area made of the thin patterned trifluoromethane (CHF₃) film was capable of functioning as a microchannel.

### EXAMPLE 2

### (1) Fabrication of a Microchannel Chip

According to the flowchart shown in FIGS. 4A and 4B, a microchannel chip was fabricated. A mask was first provided; it was a 0.025 mm-thick PET film having a score (feature size, 400 μm) cut through in a straight line, as well as a circular through-hole with an inside diameter of 5 mm being formed halfway down. This mask was placed on the upper surface of a 3 mm-thick lower substrate made of PDMS and then attached to the PDMS-made lower substrate by means of self-adsorption. The assembly was housed within a reactive ion etching apparatus and a coating of trifluoromethane (CHF₃) was applied from above the mask. After the end of the CHF3 application, the assembly was taken out of the reactive ion etching apparatus and stripped of the mask. As a result, a thin trifluoromethane (CHF₃) film with a thickness of 1 μm had been formed on the upper surface of the PDMS-made lower substrate in a pattern that replicated the mask pattern. The thin patterned trifluoromethane (CHF₃) film is an area that should serve as a non-bonding thin-film layer and, in particular, the circular non-bonding thin-film layer with a diameter of 5 mm provides an area that serves as a liquid reservoir in the microchannel chip as the final product. The upper side of the PDMS-made lower substrate having the thin patterned trifluoromethane (CHF₃) film formed on it and the lower side of a 0.1 mm-thick silicone rubber-made upper substrate having port providing through-holes with an inside diameter of 2 mm provided in specified positions were subjected to a treatment for surface modification by an oxygen plasma in the reactive ion etching apparatus. After the treatment, the lower side of the silicone rubber-made upper substrate was attached to the upper side of the PDMS-made lower substrate on which the thin patterned trifluoromethane (CHF₃) film had been formed, whereby the PDMS-made lower substrate was permanently bonded to the silicone-rubber made upper substrate. A 5 mm-thick silicone rubber-made sheet that could also be used to provide port sites was provided; it was cut through at two port sites and in a shape identical to that of the liquid reservoir (i.e., a circle with an inside diameter of 5 mm). The upper surface of the permanently bonded assembly and the lower surface of the silicone rubber-made sheet were subjected to a treatment for surface modification by an oxygen plasma in the reactive ion etching apparatus. After the treatment, the two members were attached to each other and permanently bonded.

### (2) PCR Amplification Test

The microchannel chip fabricated in (1) above was filled with a PCR solution in the liquid reservoir, and with a planar pressure being applied from above, PCR was executed to check for the occurrence of DNA amplification. First, a mixed solution (primers, DNA, dNTP, buffer, and enzyme) necessary for PCR was forced in under pressure through the port 7. As it turned out, the mixed solution forced toward the liquid reservoir site swelled to the shape of the intervening liquid reservoir site, where the liquid stayed temporarily. The liquid reservoir site expanded so much as to go beyond the circular through-hole in the upper PDMS-made sheet. Upon further forcing it under pressure, the liquid was transferred to the port 9 after a certain amount of swelling. To adjust the expanded site to the height of the surrounding area (1 mm thick), the PDMS-made sheet was compressed with a slide glass that was applied from above, whereupon the liquid was transferred to both ports, leaving in the liquid reservoir site a certain amount of the liquid that was equivalent to the thickness (1 mm) of the PDMS-made sheet. This chip was mounted in an existing PCR apparatus. The PCR apparatus had such a mechanism that an aluminum heating plate heated to 95° C. or more was pressed onto the top cover so as to prevent evaporation of the liquid inside the tube. Utilizing this mechanism, with height adjustment being made by means of an aluminum plate or the like to ensure that the whole part of the chip would be pressed from above, the plate was fixed in such a way that it would compress the entire surface of the chip with uniform force. To realize the temperature cycle of the enzyme used, temperature data was preliminarily extracted on an empirical basis and amplification was performed at optimum temperatures. As a result, using TaKaRaZ-Taq (registered trademark) of TAKARA BIO INC., a PCR cycle was completed in about 30 minutes, with DNA amplification being also verified. With the microchannel chip used in Example 2 which had the non-bonding thin-film layer having the liquid reservoir site, it was also verified that the liquid reservoir site could be utilized to have the liquid stay without blocking the ports (i.e., without sealing them) under a temperature cycle as in PCR but that the chip had only to be pressurized from above to complete the amplification job whereas after the end of the reaction, the liquid could be transferred to the port 9 by pressurizing the liquid reservoir site. In the experiment described above, once the chip was mounted in the existing PCR apparatus, the PCR mixed solution could be transferred without letting the air into the liquid reservoir site, and amplification was possible even when the PCR cycle was fast enough. These two facts show the possibility of amplification without heating the top cover to 95° C. or higher.

### EXAMPLE 3

### (1) Fabrication of a Microchannel Chip

Using the spray coating method, a microchannel chip was fabricated according to the flowchart shown in FIG. 3. A mask was first provided; it was a 0.025 mm-thick PET film having a score (feature size, 1 mm) cut through in an L-shape. This mask was placed on the upper surface of a 3 mm-thick lower substrate made of PDMS and then attached to the PDMS-made lower substrate by means of self-adsorption. From above the mask, a waterproof spray of a silicon acrylic resin-based water repellent generally available from the market was applied. After the end of spraying, the mask was removed. As a result, a coating of the silicon acrylic resin-based water repellent with a thickness of 1 μm to 5 μm had been formed on the upper surface of the PDMS-made lower substrate in an L-shaped pattern. The patterned coating of the silicon acrylic resin-based water repellent is an area that should serve as a non-bonding thin-film layer. The upper side of the PDMS-made lower substrate having the patterned coating of the silicon acrylic resin-based water repellent formed on it and the lower side of a 0.1 mm-thick silicone rubber-made upper substrate having port providing through-holes with an inside diameter of 2 mm provided in specified positions were subjected to a treatment for surface modification by an oxygen plasma in a reactive ion etching apparatus. After the treatment, the lower side of the silicone rubber-made upper substrate was attached to the upper side of the PDMS-made lower substrate on which the patterned coating of the silicon acrylic resin-based water repellent had been formed, whereby the PDMS-made lower substrate was permanently bonded to the silicone rubber-made upper substrate. A 5 mm-thick rectangular adapter having a through-hole with an inside diameter of 2 mm was permanently bonded to each of the port sites in the silicone rubber-made upper substrate after performing the same treatment for surface modification as described above.

### (2) Liquid Feeding Test

The microchannel chip fabricated in (1) above was tested for the transferability of a liquid from one port to the other. Port 9 was charged with 1 μL of the DNA staining solution Cyber Green I and microscopically examined for the occurrence of any fluorescence. Since there was no DNA available at that time, no fluorescence was observed. Port 7 was charged with 10 μL of a solution of human genome (DNA) in TE and air pressure (positive pressure) was applied to the solution in port 7 by means of a syringe connected to the through-hole in the adapter. The pressure in the port 7 was gradually increased and at the point in time when it exceeded 50 kPa, the non-bonding area that was made of the thin patterned coating of the silicon acrylic resin-based water repellent inflated to create a gap that should serve as a microchannel, through which the solution in the port 7 was transferred toward the port 9, where the DNA solution mixed with the fluorescent reagent. Examination under a fluorescence microscope showed the emission of fluorescence from the fluorescent reagent that had intercalated into the DNA. This demonstrated that the non-bonding area made of the patterned coating of the silicon acrylic resin-based water repellent by the spray coating method was capable of functioning as a microchannel.

### EXAMPLE 4

### (1) Fabrication of a Microchannel Chip

A microchannel chip of the structure shown in FIG. 1 was fabricated by the printing method. The printing side of a known conventional printing OHP (overhead projector) polyester sheet (100 μm thick) was surface modified by treatment with an oxygen plasma; thereafter, the surface-modified side was coated with an aminosilane agent so that the printing side of the OHP sheet would allow for permanent bonding. Subsequently, an L-shaped pattern drawn on a personal computer was printed on the printing side of the OHP sheet with a laser printer. Marked on the OHP sheet was a pattern of carbon black and pigment (primary component) in a thickness of 1 μm to 6 μm with a feature size of 800 μm. The upper surface of the OHP sheet where the printed thin-film pattern was marked and the lower side of an upper substrate made of a 100 μm-thick silicone rubber sheet having through-holes made to communicate with the ports 7 and 9 were surface-modified by treatment with an oxygen plasma. Subsequently, the two surface-modified sides were attached to each other, whereby the silicone rubber of the upper substrate was permanently bonded to the lower substrate made of the OHP sheet. A 5 mm-thick silicone rubber-made adapter for assisting in connection to a feed tube was secured to each site of the ports 7 and 9, to thereby fabricate the microchannel chip of the present invention.

### (2) Liquid Feeding Test

The microchannel chip fabricated in (1) above was tested for the transferability of a liquid from one port to the other. Port 9 was charged with 1 μL of the DNA staining solution Cyber Green I and microscopically examined for the occurrence of any fluorescence. Since there was no DNA available at that time, no fluorescence was observed. Port 7 was charged with 10 μL of a solution of human genome (DNA) in TE and air pressure (positive pressure) was applied to the solution in port 7 by means of a syringe connected to the through-hole in the adapter. The pressure in the port 7 was gradually increased and at the point in time when it exceeded 40 kPa, the non-bonding area that was made of the pattern of thin printed film inflated to create a gap that should serve as a microchannel, through which the solution in the port 7 was transferred toward the port 9, where the DNA solution mixed with the fluorescent reagent. Examination under a fluorescence microscope showed the emission of fluorescence from the fluorescent reagent that had intercalated into the DNA. This demonstrated that the non-bonding area made of the pattern formed by the printing method was capable of functioning as a microchannel.

### EXAMPLE 5

### (1) Fabrication of a Microchannel Chip

A microchannel chip of the structure shown in FIGS. 5A and 5B was fabricated in accordance with the method described in Example 1.

### (2) Electrophoresis Test

A gel electrophoretic substance, a polymer for use on a HITACHI microelectrophoretic apparatus, was injected through the port 7 toward the ports 9, as well as ports 7A and 7B. As a sample (analyte), DNA labelled with the fluorescent substance FITC was put into the port 7B and a voltage of 300 V was applied between port 7B and port 9B. After confirming with a fluorescence detector that the FITC-labelled DNA had reached port 9B, the voltage application between port 7B and port 9B was ceased. Subsequently, a voltage of 750 V was applied between port 7 and port 9 while a voltage of 130 V was simultaneously applied between port 7B and port 9B. At port 9, the presence of the FITC-labelled DNA could be confirmed by the fluorescence detector. This demonstrated that electrophoretic treatment could be implemented using the microchannel chip 1B of the present invention.

### EXAMPLE 6

### (1) Fabrication of a Microchannel Chip

A microchannel chip of the structure shown in FIGS. 6A and 6B was fabricated in accordance with the method described in Example 1. Note that in Example 6, primers for use in PCR were applied to form a material spotted layer on the upper surface of the non-bonding thin-film layer 11 on the lower substrate 5 and after drying the applied coating, the upper substrate 3 was permanently bonded to the lower substrate 5.

### (2) Test for Immobilizing and Holding the Material Spotted Layer

A mixed solution containing all components necessary for PCR except primers (i.e., DNA, dNTP, buffer and enzyme) was forced into port 7 under pressure. When this mixed solution reached the site of the non-bonding thin-film layer that had been coated with the primers, the dried primers mixed into the liquid chemical. The mixed solution into which the primers mixed was recovered through the port 9 and subjected to a specified PCR amplification reaction, whereupon the amplification of DNA was confirmed. This demonstrated that with the microchannel chip 1C of the present invention, PCR primers could be appropriately immobilized and held for preservation within specified regions of the channel by a technique other than binding or adsorption. From this demonstration, it can be assumed that if a coating of something like a salt or sugar having a buffering action is applied and dried in a specified area of the non-bonding thin-film layer, one needs only to feed water to prepare an optimum buffer solution within the channel.

While the microchannel chip of the present invention has been described above specifically with reference to its preferred embodiments, the present invention is by no means limited to those disclosed embodiments but various improvements and modifications are possible. For instance, the non-bonding thin-film layer 11 may be formed in a grid pattern and used in combination with a mechanism that depresses the individual crossing points until the channel is blocked and sealed; this enables the liquid to pass through the channel in many different ways. In addition, by stacking a plurality of substrates each having the non-bonding thin-film layer 11, the liquid can be fed at vertically different levels.

According to the present invention, a microchannel chip can be produced with great ease and at low cost, which contributes to a marked improvement in its practical utility and economy. As a result, the microchannel chip of the present invention finds effective and advantageous use in various fields including medicine, veterinary medicine, dentistry, pharmacy, life science, foods, agriculture, fishery, and police forensics. In particular, the microchannel chip of the present invention is optimum for use in the fluorescent antibody technique, in-situ hybridization, etc. and can be used inexpensively in a broad range of applications including testing for immunological diseases, cell culture, virus fixation, pathological test, cytological diagnosis, biopsy tissue diagnosis, blood test, bacteriologic examination, protein analysis, DNA analysis, and RNA analysis.

1. A microchannel chip comprising at least an upper substrate and a lower substrate, the upper substrate and the lower substrate being bonded together, wherein at least one non-bonding thin-film layer is formed on the bonding side of at least one substrate and at least one end portion of the non-bonding thin-film layer is connected to a port open to the atmosphere.

2. The microchannel chip according to claim 1, wherein the non-bonding thin-film layer further includes, halfway down, at least one layer of enlarged region having at least one planar shape selected from the group consisting of a circular, an elliptical, a rectangular, and a polygonal shape.

3. The microchannel chip according to claim 1, wherein the non-bonding thin-film layer is formed to provide an intersection.

4. The microchannel chip according to claim 1, wherein the non-bonding thin-film layer is formed on the bonding side of the lower substrate whereas the port is formed in the upper substrate.

5. The microchannel chip according to claim 1, wherein the non-bonding thin-film layer is formed on the bonding side of the upper substrate and the port is formed in the upper substrate.

6. The microchannel chip according to claim 1, wherein the non-bonding thin-film layer is formed on both the bonding side of the upper substrate and the bonding side of the lower substrate whereas the port is formed in the upper substrate.

7. The microchannel chip according to claim 1, wherein at least one material spotted layer is further formed in a position that corresponds to the non-bonding thin-film layer

8. The microchannel chip according to claim 7, wherein the material spotted layer is formed in a position that corresponds to the non-bonding thin-film layer and on the substrate where the non-bonding thin-film layer is not provided.

9. The microchannel chip according to claim 7, wherein the material spotted layer is formed on the non-bonding thin-film layer.

10. The microchannel chip according to claim 7, wherein the material spotted layer is formed of at least one material selected from the group consisting of chemical reaction reagents, solutes, salts, saccharides, antigens, antibodies, physiologically active substances, endocrine disrupters, sugar chains, glycoproteins, peptides, proteins, amino acids, DNAs, RNAs, microorganisms, yeasts, fungi, spores, fragmentary plant tissues, fragmentary animal tissues, drugs, glass particles, resin particles, magnetic particles, metal particles, polymers, swollen gels, and solidified gels.

11. The microchannel chip according to claim 1, wherein the upper substrate is made of polydimethyl siloxane (PDMS) whereas the lower substrate is made of polydimethyl siloxane (PDMS) or glass.

12. A process for producing the microchannel chip according to claim 1, comprising the step of applying the non-bonding thin-film layer to the bonding side of at least one of the two substrates through a mask having a desired through-pattern by either one of commonly employed chemical thin-film forming methods.

13. The process for producing the microchannel chip according to claim 1, comprising the step of applying the non-bonding thin-film layer to the bonding side of at least one of the two substrates through a mask having a desired through-pattern by spraying a coating agent.

14. The process for producing the microchannel chip according to claim 1, wherein the non-bonding thin-film layer is formed by printing on
the bonding side of at least one of the two substrates.
